(19) United States
(12) Patent Application Publication
(10) Pub. No.: US 2026/0311352 A1
(43) Pub. Date: Oct. 8, 2026

(54) MEASUREMENT OF SURFACE ROUGHNESS

(71) Applicant: TRINAMIX GMBH, Ludwigshafen am Rhein (DE)

(72) Inventors: Jalpa PARMAR, Ludwigshafen am Rhein (DE); Christian LENNARTZ, Ludwigshafen am Rhein (DE); Stephan KNAPP, Ludwigshafen am Rhein (DE); Peter FEJES, Dornbirn (AT)

(21) Appl. No.: 19/485,323

(22) PCT Filed: May 27, 2024
(86) PCT No.: PCT/EP2024/064528

§ 371 Date: Nov. 18, 2025

(30) Foreign Application Priority Data
Jun. 2, 2023 (EP) 23176916.7

(51) Int. Cl. G06T 7/40; A61B 5/00; G06T 7/00; H04N 23/21; H04N 23/56

(52) U.S. Cl. CPC G06T 7/40; A61B 5/0082; A61B 5/442; G06T 7/0012; H04N 23/21; H04N 23/56; A61B 2576/02; G06T 2207/10048; G06T 2207/30088

(57) ABSTRACT

Disclosed herein is a method for measuring a surface roughness associated with an object, the method comprising including:
a) receiving a speckle image showing an object while being illuminated by coherent electromagnetic radiation associated with a wavelength between 850 nm and 1400 nm,
b) determining a surface roughness measure based on the speckle image, and
c) providing the surface roughness measure.

### TECHNICAL FIELD

The invention relates to a method for measuring a surface roughness, a non-transitory computer-readable storage medium, use of a surface roughness measure, a device or system for measuring a surface roughness.

### TECHNICAL BACKGROUND

Currently measurement of surface roughness use light in the visible range. These setups require fixed setups. Often direct contact and experts for carrying out the measurement are needed.

Alternatively, conductance-based measurements are deployed providing inaccurate measurement results while requiring contact with the surface to be measured.

Hence, there is a desire to provide easy, reliable and contact-free methods and devices for measuring the surface roughness.

### SUMMARY

In one aspect, a method for measuring a surface roughness associated with an object is disclosed, the method includes a) receiving a Speckle image showing an object while being illuminated by coherent electromagnetic radiation associated with a wavelength between 850 nm and 1400 nm, b) determining a surface roughness measure based on the Speckle image, c) providing the surface roughness measure.

In one aspect, a non-transitory computer-readable storage medium is disclosed, the computer-readable storage medium including instructions that when executed by a computer, cause the computer to a) receive a Speckle image showing an object under illumination with coherent electromagnetic radiation associated with a wavelength between 850 nm and 1400 nm, b) determine a surface roughness measure based on the Speckle image, c) provide the surface roughness measure.

In one aspect, use of a surface roughness measure for evaluating the surface roughness of a human is disclosed.

In one aspect, a device or system for measuring a surface roughness is disclosed, the system or device includes an illumination source configured for illuminating an object with coherent electromagnetic radiation associated with a wavelength between 850 nm and 1400 nm, a camera configured for generating a Speckle image showing an object under illumination with coherent electromagnetic radiation associated with a wavelength between 850 nm and 1400 nm, a processor configured for receiving the Speckle image from the camera and determining a surface roughness measure based on the Speckle image and providing the surface roughness measure.

Any disclosure, embodiments and examples described herein relate to the methods, the systems, apparatuses, chemical products and computer elements lined out above and below. Advantageously, the benefits provided by any of the embodiments and examples equally apply to all other embodiments and examples.

### EMBODIMENTS

In the following, terminology as used herein and/or the technical field of the present disclosure will be outlined by ways of definitions and/or examples. Where examples are given, it is to be understood that the present disclosure is not limited to said examples.

Skin roughness constitutes an essential parameter for assessing skin health. Currently skin roughness is estimated inaccurately and/or under artificial conditions. Often a direct contact with the skin is required leading to distraction of the human whose skin is assessed. This may be unpleasant to the human and result in a raise of the respective stress level. For example, the raised stress level may result in the production of sweat which in turn disturbs the measurement of the surface roughness. Furthermore, experts and advanced, high-cost technology are required to carry out the measurements. Hence, there is a need for low-cost, easy, reliable and non-invasive measurement of surface roughness.

By deploying the methods, devices and systems as described herein, the surface roughness can be measured easily with low-cost hardware. Such hardware is readily available and can be integrated easily in mobile electronic devices such as smartphone. Furthermore, resources for time consuming measurements are saved. Hence, determining a surface roughness measure based on a speckle image showing the object such as a human while being illuminated by coherent electromagnetic radiation associated with a wavelength between 850 nm and 1400 nm enables mobile electronic devices to measure surface roughness. These devices can be operated by the non-expert user of the mobile electronic device enabling thus, measurements under more natural conditions. Furthermore, the wavelength of the coherent electromagnetic radiation is invisible to the human and thus, the human and specifically the eyes are not distracted or disturbed by the measurement. Followingly, the measurement can be carried out in the darkness. Another benefit is that the measurement of the surface roughness can be performed under ambient light. As the wavelength of the coherent electromagnetic radiation is chosen such that the contribution of the ambient light to the intensity signal associated with the speckle image can be eliminated and/or neglected. Since the measurement of the surface roughness is based the speckle image, no contact to the skin of the human is needed and a contact-free operation is enabled while the analysis of the speckle image for determining the surface roughness measure is fast.

In an embodiment, Camera may refer, without limitation, to a device having at least one image sensor configured for generating or recording spatially resolved one-dimensional, two-dimensional or even three-dimensional optical data or information. The camera may be a digital camera. As an example, the camera may comprise at least one image sensor, such as at least one CCD sensor and/or at least one CMOS sensor configured for recording images.

In an embodiment, the camera may comprise an image sensor, a lens. Preferably, the camera may comprise an image sensor, a lens and a polarizer. A lens may refer to an optical element suitable for influencing the expansion of the light beam associated with the coherent electromagnetic radiation. The polarizer may refer to an optical element suitable for selecting the electromagnetic radiation according to its polarization. In particular, the polarizer may refer to an optical element suitable for selecting the coherent electromagnetic radiation according to polarization its. Followingly, a part of the electromagnetic radiation, in particular coherent electromagnetic radiation may pass the polarizer while the rest of the electromagnetic radiation, in particular coherent electromagnetic radiation, may be turned away at least partially and/or may be absorbed at least partially.

As the coherent electromagnetic radiation associated with a wavelength between 850 nm and 1400 nm penetrates the skin deeply, a part of the information received from the light reflected from the skin comprises information independent from the surface roughness which distracts the measurement of the surface roughness. To increase the signal-to-noise ratio a polarizer can be used. The coherent electromagnetic radiation reflected from the surface of the object is usually polarized differently as the light reflected from deeper layers of the human skin. Followingly, the polarizer enables a selection of the desired signal from the undesired signal.

In an embodiment, Coherent electromagnetic radiation may refer to electromagnetic radiation that is able to exhibit interference effects. It may also include partial coherence, i.e. a non-perfect correlation between phase values. Preferably, the coherent electromagnetic radiation may be in the infrared range. The coherent electromagnetic radiation is associated with a wavelength between 850 nm and 1400 nm. Preferably, the coherent electromagnetic radiation may be associated with a wavelength between 880 nm and 1300 nm. In an embodiment, the coherent electromagnetic radiation may be associated with a wavelength between 900 nm and 1000 nm and/or wherein the coherent electromagnetic radiation may be associated with a wavelength between 1100 nm and 1200 nm and/or wherein the coherent electromagnetic radiation may be associated with a wavelength between 1340 nm and 1440 nm. This is advantageous since the sunlight has a bandgap in these regions. Followingly, the coherent electromagnetic radiation with the abovementioned wavelengths illuminated for generating the speckle image can be differentiated easier from incoming sun light. Followingly, the used of coherent electromagnetic radiation within the above-specified region enables measurements of surface roughness with the presence of sun light such as in the nature. Consequently, measurements of surface roughness can be easily and location-independently used. Overall, an improved signal-to-noise ratio is achieved and the accuracy of evaluations of surface roughness is increased.

In an embodiment, computer-readable storage medium may refer to any suitable data storage device or computer readable memory on which is stored one or more sets of instructions (for example software) embodying any one or more of the methodologies or functions described herein. The instructions may also reside, completely or at least partially, within the main memory and/or within the processor during execution thereof by the computer, main memory, and processing device, which may constitute computer-readable storage media. The instructions may further be transmitted or received over a network via a network interface device. Computer-readable storage medium includes hard drives, for example on a server, USB storage device, CD, DVD or Blue-ray discs.

In an embodiment, the speckle pattern may refer to a distribution of the plurality of speckles. The distribution of the plurality of speckles may refer to a spatial distribution of at least one of the plurality of speckles and/or a spatial distribution of at least two of the plurality of speckles in relation to each other. Spatial distribution of the at least one of the plurality of speckles may refer to and/or specify a spatial extent of the at least one of the plurality of speckles. Spatial distribution of the at least two of the plurality of speckles may refer to and/or specify a spatial extent of the first speckle of the at least two speckles in relation to the second speckle of the at least two speckles and/or a distance between the first speckle of the at least two speckles and the second speckle of the at least two speckles.

In an embodiment, Illumination source may refer to a device suitable for illuminating the object by coherent electromagnetic radiation and/or suitable for emitting coherent electromagnetic radiation. The illumination source may comprise at least one radiation source. The illumination source may comprise a plurality of radiation sources. The illumination source may comprise for example at least one laser source and/or at least one semiconductor radiation source. A semiconductor radiation source may be for example a light-emitting diode such as an organic, a laser diode and/or inorganic light-emitting diode. Additionally or alternatively, the radiation source may be a VCSEL array and/or a LED. Additionally or alternatively, the illumination source may comprise a VCSEL array and/or a LED. Further, the illumination source may comprise one or more optical elements. Optical element may be for example a lens, a metasurface element, a DOE or a combination thereof. Hence, an illumination source may comprise one or more radiation sources and one or more optical elements.

In an embodiment, camera may refer to at least one unit of the optoelectronic apparatus configured for generating at least one image. The image may be generated via a hardware and/or a software interface, which may be considered as the camera. The camera may comprise at least one image sensor, in particular at least one pixelated image sensor. The camera may comprise at least one CMOS sensor and/or at least one CCD chip. For example, the camera may comprise at least one CMOS sensor, which may be sensitive in the infrared spectral range. The camera may have a field of view between 10°×10° and 75°×75°, preferably 55°×65°. The camera may have a resolution below 2 megapixel, preferably between 0.3 megapixel and 1.5 megapixel. Megapixel may refer to a unit for measuring the number of pixels associated with a camera and/or an image. The camera may comprise further elements, such as one or more optical elements, e.g. one or more lenses. As an example, the optical sensor may be a fix-focus camera, having at least one lens which is fixedly adjusted with respect to the camera. Alternatively, however, the camera may also comprise one or more variable lenses which may be adjusted, automatically or manually. Other cameras, however, are feasible.

In an embodiment, Model may be suitable for determining an output based on an input. In particular, model may be suitable for determining a surface roughness measure based on the speckle image, preferably based on receiving speckle image. A model may be a physical model, a data-driven model or a hybrid model.

A hybrid model may be a model comprising at least one data-driven model with physical or statistical adaptations and model parameters. Statistical or physical adaptations may be introduced to improve the quality of the results since those provide a systematic relation between empiricism and theory.

In an embodiment, data-driven model may represent a correlation between the surface roughness measure and the speckle image. The data-driven model may obtain the correlation between surface roughness measure and speckle image based on a training data set comprising a plurality of speckle images and a plurality of surface roughness measures.

In an embodiment, data-driven model may be parametrized based on a training data set to receive the speckle image and provide the surface roughness measure. The data-driven model may be trained based on a training data set. The training data set may comprise at least one speckle image and at least one corresponding surface roughness measure. The training data set may comprise a plurality of speckle image and a plurality of surface roughness measures. Training the model may include parametrizing the model. The data-driven model may be parametrized and/or trained to provide the surface roughness measure based on the speckle image, in particular receiving the speckle image. Determining the surface roughness measure based on the speckle image may comprise providing the speckle image to a data-driven model and receiving the surface roughness measure from the data-driven model. Providing surface roughness measure based on the speckle image may comprise mapping the speckle image to the surface roughness measure. The data-driven model may be parametrized and/or trained to receive the speckle image. Data-driven model may receive the speckle image at an input layer.

The term training may also be denoted as learning. The term specifically may refer, without limitation, to a process of building the data-driven model, in particular determining and/or updating parameters of the data-driven model. Updating parameters of the data-driven model may also be referred to as retraining. Retraining may be included when referring to training herein. During the training the data-driven model may adjust to achieve best fit with the training data, e.g. relating the at least on input value with best fit to the at least one desired output value. For example, if the neural network is a feedforward neural network such as a CNN, a backpropagation-algorithm may be applied for training the neural network. In case of a RNN, a gradient descent algorithm or a backpropagation-through-time algorithm may be employed for training purposes. Training a data-driven model may include or may refer without limitation to calibrating the model.

In an embodiment, physical model may reflect physical phenomena in mathematical form, e.g., including first-principles models. A physical model may comprise a set of equations that describe an interaction between the object and the coherent electromagnetic radiation thereby resulting in a surface roughness measure. The physical model may be based on at least one of a fractal dimension, speckle size, speckle contrast, speckle modulation, roughness exponent, standard deviation of the height associated with surface features, lateral correlation length, average mean height, root mean square height or a combination thereof. In particular, the physical model may comprise one or more equations relating the speckle image and the surface roughness measure based on equations relating to the fractal dimension, speckle size, speckle contrast, speckle modulation, roughness exponent, standard deviation of the height associated with surface features, lateral correlation length, average mean height, root mean square height or a combination thereof.

In an embodiment, Object may refer to a physical entity. Object may comprise one or more materials. Materials may include skin, in particular a human's skin, paper, wood, glass, metal oxides such as aluminium oxide, leather or the like. In particular, object may refer to a living organism. Preferably, the object may be a human. Where the object is a living organism such as a human, at least a part of the skin of the living organism such as a human may be illuminated with the coherent electromagnetic radiation.

In an embodiment, the object may be a human and the surface roughness may be associated with the skin of the human. For this purpose, the speckle image may show at least a part of the skin of the human while being illuminated by coherent electromagnetic radiation.

In an embodiment, patterned coherent electromagnetic radiation may refer to a plurality of light beams of coherent electromagnetic radiation. Patterned coherent electromagnetic radiation may comprise a plurality of light beams, eg at least two light beams, preferably at least two light beams. Projection of a light beam of the patterned coherent electromagnetic radiation onto a surface may result in a light spot. Followingly, projecting a plurality of light beams of the patterned coherent electromagnetic radiation onto the object may result in a plurality of light spots on the object. Preferably, the number of light spots may be equal to the number of light beams associated with the patterned coherent electromagnetic radiation. The one or more light spots may be shown in the speckle image. A projection of patterned coherent electromagnetic radiation onto a regular surface may result in a light spot projected onto the regular surface independent of speckle. A projection of patterned coherent electromagnetic radiation onto a regular surface may result in a light spot projected onto the irregular surface comprising at least one speckle, preferably a plurality of speckle. Object may be associated with an at least partially irregular surface. Followingly, the speckle image may comprise a plurality of speckle. In particular, if the object may comprise at least partially of skin, a plurality of speckle is formed due to the interference of the coherent electromagnetic radiation. Followingly, a light spot may comprise zero, one or more speckle depending on the surface the patterned coherent electromagnetic radiation is projected on. Skin may have an irregular surface. Hence the projection of patterned coherent electromagnetic radiation may result in the formation of speckle within the one or more light spots. A light spot may be a result of the projection of a light beam associated with the patterned coherent electromagnetic radiation. A light spot refers to an arbitrarily shaped spot of coherent electromagnetic radiation. A light spot may refer to a contiguous area illuminated with coherent electromagnetic radiation. Projecting coherent electromagnetic radiation on an irregular surface results in the formation of speckle. Followingly, the light spot may comprise one or more speckle. A light spot may have a diameter between 0.5 mm and 5 cm, preferably 0.6 mm and 4 cm, more preferably, 0.7 mm and 3 cm, most preferably 0.4 and 2 cm.

For example, patterned coherent electromagnetic radiation may be generated by an illumination source comprising a plurality of light emitters such as a VCSEL array comprises a plurality of VCSELs. An emitter of the plurality of light emitters may emit one light beam. Hence, an emitter of the plurality of light emitters may be associated with the one light spot, with the formation of one light spot and/or with the projection of one light spot. Additionally or alternatively, patterned coherent electromagnetic radiation may be generated by one or more light emitters and an optical element such as a DOE or a metasurface element. A metasurface element may be a meta lense. A meta lense may be at least partially transparent with respect to the coherent electromagnetic radiation and/or may comprise a material associated with a structure on the nanoscale. The optical element may replicate the number of light beams associated with the one or more light emitters and/or may be suitable for replicating the number of light beams associated with the one or more light emitters. For example, the light emitter may be a laser.

Additionally or alternatively, patterned coherent electromagnetic radiation may be generated by one or more light emitters and an optical element such as a DOE or a metasurface element. The optical element may replicate the number of light beams associated with the one or more light emitters and/or may be suitable for replicating the number of light beams associated with the one or more light emitters. For example, the light emitter may be a laser.

In an embodiment, processor may refer to an arbitrary logic circuitry configured to perform basic operations of a computer or system, and/or, generally, to a device which is configured for performing calculations or logic operations. In particular, the processor, or computer processor may be configured for processing basic instructions that drive the computer or system. It may be a semi-conductor based processor, a quantum processor, or any other type of processor configures for processing instructions. As an example, the processor may be or may comprise a Central Processing Unit (“CPU”). The processor may be a (“GPU”) graphics processing unit, (“TPU”) tensor processing unit, (“CISC”) Complex Instruction Set Computing microprocessor, Reduced Instruction Set Computing (“RISC”) microprocessor, Very Long Instruction Word (“VLIW”) microprocessor, or a processor implementing other instruction sets or processors implementing a combination of instruction sets. The processing means may also be one or more special-purpose processing devices such as an Application-Specific Integrated Circuit (“ASIC”), a Field Programmable Gate Array (“FPGA”), a Complex Programmable Logic Device (“CPLD”), a Digital Signal Processor (“DSP”), a network processor, or the like. The methods, systems and devices described herein may be implemented as software in a DSP, in a micro-controller, or in any other side-processor or as hardware circuit within an ASIC, CPLD, or FPGA. It is to be understood that the term processor may also refer to one or more processing devices, such as a distributed system of processing devices located across multiple computer systems (e.g., cloud computing), and is not limited to a single device unless otherwise specified. The processor may also be an interface to a remote computer system such as a cloud service. The processor may include or may be a secure enclave processor (SEP). An SEP may be a secure circuit configured for processing the spectra. A “secure circuit” is a circuit that protects an isolated, internal resource from being directly accessed by an external circuit. The processor may be an image signal processor (ISP) and may include circuitry suitable for processing images, in particular images with personal and/or confidential information.

In an embodiment, Speckle may refer to an optical phenomenon caused by interfering coherent electromagnetic radiation due to non-regular or irregular surfaces. Speckles may appear as contrast variations in an image such as a speckle image.

In an embodiment, Speckle image may refer to an image showing a plurality of speckles. The speckle image may show a plurality of speckles. The speckle image may be generated while the object may be illuminated by coherent electromagnetic radiation associated with a wavelength between 850 nm and 1400 nm. The speckle image may show a speckle pattern. The speckle pattern may specify a distribution of the speckles. The speckle image may indicate the spatial extent of the speckles. The speckle image may be suitable for determining a surface roughness measure. The speckle image may be generated with a camera. For generating the speckle image, the object may be illuminated by the illumination source.

In an embodiment, Surface feature may refer to an arbitrarily shaped structure associated with the surface of the object. In particular, the surface feature may refer to a substructure of the surface associated with the object. A surface may comprise a plurality of surface features. For example, an uplift or a sink may be surface features. Preferably, a surface feature may refer to a part of the surface associated with an angle unequal to 90° against the surface normal.

In an embodiment, Surface roughness may refer to a property of a surface associated with the object. In particular, the surface roughness may characterize lateral and/or vertical extent of surface features. The surface roughness may be evaluated based on the surface roughness measure. The surface roughness measure may quantify the surface roughness. For example, the surface

In an embodiment, Surface roughness measure may refer to a measure suitable for quantifying the surface roughness. Surface roughness measure may be related to the speckle pattern. For example, surface roughness measure may include at least one of a fractal dimension, speckle size, speckle contrast, speckle modulation, roughness exponent, standard deviation of the height associated with surface features, lateral correlation length, average mean height, root mean square height or a combination thereof. Preferably, the surface roughness measure may be suitable for describing the vertical and lateral surface features. Surface roughness measure may comprise a value associated with the surface roughness measure. Surface roughness measure may refer to a term of a quantity for measuring the surface roughness and/or to the values associated with the quantity for measuring the surface roughness.

In an embodiment, the fractal dimension may be determined based on the Fourier transform of the speckle image and/or the inverse of the Fourier transform of the speckle image. In particular, the fractal dimension may be determined based on the slope of a linear function fitted to a double logarithmic plot of the power spectral density versus a frequency obtained by Fourier transform. Speckle size may refer to the spatial extent of one or more speckles. Where the speckle size may refer to the spatial extent of more than one speckle, the speckle size may be determined based on an average of more than one speckle sizes and/or a weighting of the more than one speckle sizes. Speckle contrast may refer to a measure for the standard deviation of at least a part of the speckle image in relation to the mean intensity of at least the part of the speckle image. The speckle modulation may refer to a measure for the intensity fluctuation associated with the speckles in at least a part of the speckle image. Roughness exponent, standard deviation of the height associated with surface features, lateral correlation length or a combination thereof may be determined based on the autocorrelation function associated with the double logarithmic plot of the power spectral density versus a frequency obtained by Fourier transform.

In an embodiment, determining the surface roughness measure based on the speckle image may refer to determining the surface roughness measure based on a speckle pattern. Determining the surface roughness based on the speckle pattern may refer to determining the surface roughness based on a distribution of a plurality of speckles in the speckle image. Determining the surface roughness measure based on the distribution of the plurality of speckles in the speckle image may refer to determining the distribution of the plurality of speckles in the speckle image. Determining the distribution of the speckles may comprise determining at least one of fractal dimension associated with the speckle image, speckle size associated with the speckle image, speckle contrast associated with the speckle image, speckle modulation associated with the speckle image, roughness exponent associated with the speckle image, standard deviation of the height associated with surface features associated with the speckle image, lateral correlation length associated with the speckle image, average mean height associated with the speckle image, root mean square height associated with the speckle image or a combination thereof. Additionally or alternatively, determining the surface roughness measure may comprise determining at least one of fractal dimension associated with the speckle image, speckle size associated with the speckle image, speckle contrast associated with the speckle image, speckle modulation associated with the speckle image, roughness exponent associated with the speckle image, standard deviation of the height associated with surface features associated with the speckle image, lateral correlation length associated with the speckle image, average mean height associated with the speckle image, root mean square height associated with the speckle image or a combination thereof. As the speckles are caused by the irregularities of the surface, the speckles reflect the roughness of the surface. Followingly, determining the surface roughness measure based on the speckles in the speckle image utilizes the relation between the speckle distribution and the surface roughness. Thereby, a low-cost, efficient and readily available solution for surface roughness evaluation apart from fixed medical or cosmetic context is enabled.

In an embodiment, the coherent electromagnetic radiation may be patterned coherent electromagnetic radiation and/or wherein the coherent electromagnetic radiation may comprise one or more light beams. Preferably, the coherent electromagnetic radiation may comprise at least two, more preferably at least 5 light beams. Patterned coherent electromagnetic radiation may refer to coherent electromagnetic radiation being associated with more than one light beam and/or comprising more than one light beam. One light beam may illuminate at least a part of the object and/or may be associated with a contiguous area of coherent electromagnetic radiation on at least a part of the object. A light spot may refer to the contiguous area of coherent electromagnetic radiation on at least a part of the object. Followingly, the object being illuminated by patterned coherent electromagnetic radiation may be illuminated by a plurality of light spots. The light spots may be overlapping at least partially. The intensity associated with a light spot may be substantially similar. Substantially similar may refer to intensity values associated with the light spot may differ by less than 50%, preferably less than 30%, more preferably less than 20%. Using patterned light is advantageous since it enables the sparing of light-sensitive regions such as the eyes.

In an embodiment, determining the surface roughness measure may be based on the distribution of the speckles in the speckle image. Determining the surface roughness measure based on the distribution of the speckles in the speckle image may comprise determining at least one of a size distribution of the speckles, a power spectral density associated with the speckle image, a fractal dimension associated with the speckle image, a speckle contrast, a speckle modulation or a combination thereof. Additionally or alternatively, determining the surface roughness measure based on the distribution of the speckles in the speckle image may comprise providing the speckle image to a model, in particular a data-driven model, wherein the data-driven model may be parametrized and/or trained based on a training data set comprising one or more speckle image and one or more corresponding surface roughness measure.

In an embodiment, the distance between the object and a camera used for generating the speckle image may be between 10 cm and 1.5 m and/or wherein the distance between the object and an illumination source used for illuminating the object may be between 10 cm and 1.5 m. Preferably, the distance between the object and the camera may be between 20 cm and 1.2 m. Preferably, the distance between the object and the illumination source may be between 20 cm and 1.2 m. Adjusting the distance between object and camera ensure that a speckle image of sufficient quality is generated. Followingly, the above-specified distances enable a correct and reliable determination of the surface roughness. This is especially important in non-static contexts, where a user may operate the device by himself.

In an embodiment, the speckle image may show a human while being illuminated by coherent electromagnetic radiation and the surface roughness of a human may be determined. Preferably, the human may have generated the speckle image and/or initiated the generation of the speckle image. Preferably, the speckle image may be initiated by the human operating an application of a mobile electronic device. By doing so, the human can decide on his or her own when to determine the surface roughness of her or his skin. Followingly, a nonexpert user is enabled to determine surface roughness and measurements can be carried out in more natural and less artificial contexts. Thereby, the surface roughness can be evaluated more realistically which in turn serves more realistic measure for the surface roughness. For example, the skin may have different surface roughness during the course of the day depending on the activity of the human. Doing sports may influence the surface roughness as well as creaming the skin. This influence can be verified with the herein described methods and systems.

In an embodiment, the surface roughness measure may be determined by a mobile electronic device and/or wherein the speckle image is generated with a camera of a mobile electronic device. In particular, the human may initiate the generation of the speckle image based on the mobile electronic device. This is beneficial since many humans own a mobile electronic device such as a smartphone. These devices accompany the human and thus, a measurement of the surface roughness is possible at any time and can be carried out in more natural and less artificial contexts. Thereby, the surface roughness can be evaluated more realistically which in turn serves more realistic measure for the surface roughness.

In an embodiment, the surface roughness measure may be determined based on the speckle image by providing the speckle image to a model and receiving the surface roughness measure from the model. The model may be a data-driven model and may be parametrized and/or trained based on a training data set comprising a plurality of speckle images and corresponding surface roughness measures or indications of surface roughness measures. Additionally or alternatively, the model may be a physical model.

In an embodiment, the speckle image may be associated with a resolution of less than 5 megapixel. Preferably, the speckle image may be associated with a resolution of less than 3 megapixel, more preferably less than 2.5 megapixel, most preferably less than 2 megapixel.

Such speckles images can be generated with readily available, small and cheap smartphone cameras. Furthermore, the storage and processing capacities needed for evaluating the surface roughness measure are small. Thus, the low resolution of the speckle image used for evaluating the surface roughness enables the usage of mobile electronic devices for evaluating the surface roughness, in particular devices like smartphone or wearables since these devices have strictly limited size, memory and processing capacity.

In an embodiment, image augmentation techniques may comprise at least one of scaling, cutting, rotating, blurring, warping, shearing, resizing, folding, changing the contrast, changing the brightness, adding noise, multiply at least a part of the pixel values, drop out, adjusting colors, applying a convolution, embossing, sharpening, flipping, averaging pixel values or the like.

In an embodiment, the method may further include and/or the processor may be further configured for reducing the speckle image to a predefined size prior to determining the surface roughness measure. Reducing the speckle image to a predefined size may be based on applying one or more image augmentation techniques. Reducing the speckle image to a predefined sized may comprise selecting an area of the speckle image of the predefined size and cutting the speckle image to the area of the speckle image of the predefined size. The area of the speckle image of the predefined size may be associated with the living organism such as the human, in particular with the skin of the living organism such as the skin of the human. The part of the image other than the area of the speckle image of the predefined size may be associated with background and/or may be independent of the living organism such as a human. By doing so a reduced amount of data needs to be processed which decreases the time needed for determining the surface roughness or allows for less storage and processor to be needed. Furthermore, the part of the image useful for the analysis is selected. Hence, reducing the size may result in disregarding parts of the speckle image independent of the object or living organism such as a human. Followingly, the surface roughness measure can be determined easily and for the analysis disturbing parts not relating to the object are ignored.

In an embodiment, the method may further include and/or the processor may be further configured for reducing the speckle image to a predefined size based on detecting the object in the speckle image. Preferably, the method may further include and/or the processor may be further configured for reducing the speckle image to a predefined size based on detecting the user in the speckle image. Preferably, the speckle image may be reduced to a predefined size based on detecting the object in the speckle image prior to determining the surface roughness measure. In particular, the speckle image may be reduced to a predefined size based on detecting the user in the speckle image prior to determining the surface roughness measure. Reducing the speckle image to the predefined size based on detecting the object in the speckle image may include detecting the contour of the object and reducing the speckle image to an area associated with the object. In particular, reducing the speckle image to the predefined size based on detecting the user in the speckle image may include detecting the contour of the user, eg detecting the contour of a user's face and reducing the speckle image to an area associated with the user, in particular with an area associated with the user's face. Preferably the area associated with the user may be within the contour of the object, in particular the user and/or the contour of a user's face. For this purpose, the method may further include and/or the processor may be further configured for receiving a flood image. The flood image may be generated while the object may be illuminated by flood illumination. The flood image may show the contour of the object, in particular the contour of the user. The contour of the object, in particular of the user may be detected based on the flood image. Preferably, the contour of the object, in particular of the user may be detected by providing the flood image to an object detection data-driven model, in particular a user detection model, wherein object detection data-driven model may be parametrized and/or trained to receive the flood image and provide an indication on the contour of the object based on a training data set comprising flood images and indications on the contour of the objects. The indication of the contour of the object may include a plurality of points indicating the location of a specific landmark associated with the object. For example, where the speckle image may be associated with a user's face, the user's face may be detected based on the contour, wherein the contour may indicate the landmarks of the face such as the nose point or the outer corner of the lips or eyebrows.

In an embodiment, the method may further include and/or the processor may be further configured for generating a partial speckle image. A partial speckle image may refer to a partial image generated based on the speckle image. The partial speckle image may be generated by applying one or more image augmentation techniques to the speckle image.

In an embodiment, method may further include and/or the processor may be further configured for generating a first speckle image and a second speckle image. The speckle image may comprise the first speckle image and the second speckle image. The first speckle image may refer to a first part of the speckle image. The second speckle image may refer to a second part of the speckle image. Preferably, the first speckle image and the second speckle image be different from each other. In particular, the first speckle image and the second speckle image may be non-overlapping. The first speckle image and the second speckle image may be generated by applying one or more image augmentation techniques to the speckle image. Determining the surface roughness measure based on the speckle image may comprise determining a first surface roughness measure based on the first speckle image and determining a second surface roughness measure based on the second speckle image. Providing the surface roughness measure may include providing the first surface roughness measure and the second surface roughness measure. In particular, the first surface roughness measure and the second surface roughness measure may be provided together. Preferably, the first surface roughness measure and the second surface roughness measure may be provided in a surface roughness measure map indicating the spatial distribution of surface roughness measures. For example, the surface roughness measure map may indicate the first surface roughness measure associated with a first area in the surface roughness measure map and the second surface roughness measure map may indicate the second surface roughness measure associated with a second area in the surface roughness measure map. In particular, the surface roughness measure map may be similar to a heat map, wherein the surface roughness measures may be plotted against the area associated with the respective surface roughness measures.

In an embodiment, the surface roughness measure may be used for evaluating the surface roughness of a human's skin.

These and other objects, which become apparent upon reading the following description, are solved by the subject matters of the independent claims. The dependent claims refer to embodiments of the invention.

### BRIEF DESCRIPTION OF THE DRAWINGS

In the following, the present disclosure is further described with reference to the enclosed figures. The same reference numbers in the drawings and this disclosure are intended to refer to the same or like elements, components, and/or parts.

FIG. 1 illustrates an example system for measuring a surface roughness.

FIG. 2 illustrates an example for determining a surface roughness measure.

FIG. 3 illustrates an example embodiment of a method for measuring a surface roughness associated with an object 300.

FIG. 4 illustrates an embodiment of a surface associated with an object.

### DETAILED DESCRIPTION

The following embodiments are mere examples for implementing the method, the system or application device disclosed herein and shall not be considered limiting.

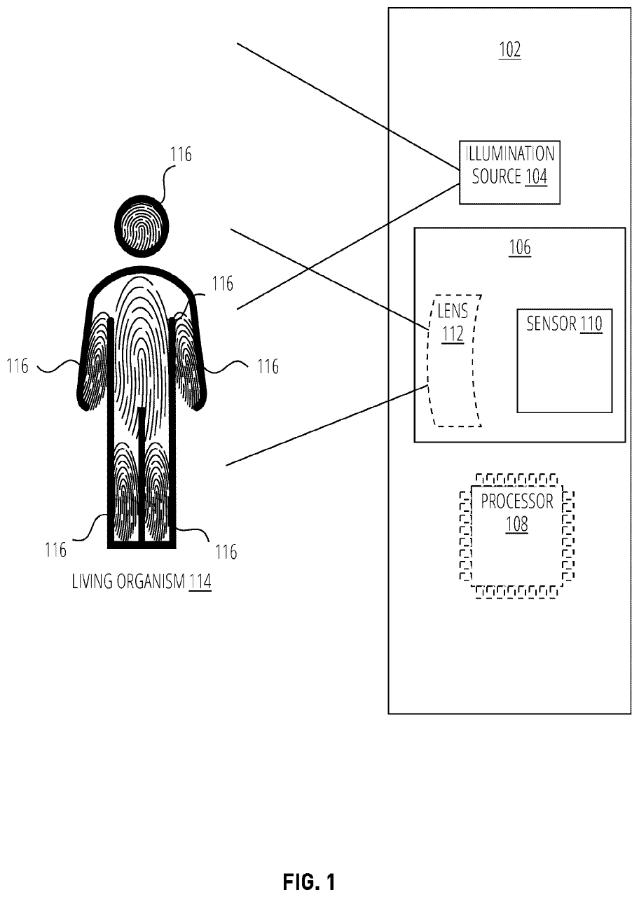

FIG. 1 illustrates an example system for measuring a surface roughness. The system comprises an Illumination source 104, a 106 and a processor 108. The surface roughness may be determined with respect to an object such as a human. Preferably, the object may be a living organism 114 such as a human. The living organism 114 may have skin. The skin may be associated with a surface roughness. The surface roughness can be evaluated based on a surface roughness measure. The skin may be exposed to coherent electromagnetic radiation associated with a wavelength between 850 nm and 1400 nm.

The coherent electromagnetic radiation associated with a wavelength between 850 nm and 1400 nm may be emitted by the illumination source 104. The illumination source 104 may comprise one or more radiation sources such as a VCSEL array or a single laser diode. The radiation source may be associated with one or more light beams. For example, a single laser diode may emit one light beam, whereas a VCSEL array may emit a plurality of light beams. Preferably the number of light beams correspond to the number of VCSELs in the VCSEL array. Thus, the illumination source may emit a plurality of light beams. The plurality of light beams may result in projecting a pattern onto the object. Preferably, the illumination source 104 may emit patterned coherent electromagnetic radiation. Patterned coherent electromagnetic radiation may be suitable for projection a pattern onto the object. Additionally or alternatively, the illumination source 104 may comprise one or more optical elements. An optical element may be suitable for splitting and/or multiplicating light beams. Examples for optical elements can be diffractive optical elements, refractive optical elements, meta surface elements, lenses or the like. Hence, an illumination source 104 comprising a single laser diode or a VCSEL array in combination with an optical element may result in illuminating the object with patterned coherent electromagnetic radiation. The illumination source 104 may be associated with a field of illumination as indicated by the two lines originating from the illumination source 104.

Depending on the body part illuminated by the coherent electromagnetic radiation, different skin surface roughness measures may be determined. Different body parts of the living organism 114 may be associated with different skin roughness 116. For example, a hand may be associated with a higher skin roughness whereas the face may be associated with a lower skin surface roughness. The surface roughness may be characteristic for a body part of the living organism 114 and/or for the identity of the living organism 114.

A Speckle image may be generated while the living organism 114 may be illuminated with coherent electromagnetic radiation, preferably patterned coherent electromagnetic radiation. Coherent electromagnetic radiation may interact with the skin of the living organism 114 once it may be projected onto the skin of the living organism 114. Coherent electromagnetic radiation forms speckle when interacting with a non-homogeneous and uneven surface such as skin. The different wavefronts of the coherent electromagnetic radiation may interact by means of interference. The interference of the different wavefronts of the coherent electromagnetic radiation may result in contrast variations of the coherent electromagnetic radiation on the skin of the living organism 114. These contrast variations may depend on the surface roughness associated with the surface the coherent electromagnetic radiation is illuminating. Hence, the roughness associated with the skin may influence the formation of speckle such as the size and orientation of the speckle. Followingly, analysis of the speckle may result in a surface roughness measure.

For analyzing the speckle, a Speckle image is generated with a 106 such as a camera. The 106 may comprise a sensor 110. Optionally, the 106 may comprise a lens 112. In another example, the 106 may comprise a polarizer. The coherent electromagnetic radiation is in the infrared range. Thus, the coherent electromagnetic radiation may penetrate deeper than the epidermis. The surface roughness measure may specify the surface roughness associated with the surface of skin. Followingly, information obtained by coherent electromagnetic radiation penetrating for example the dermis or deeper may overlay the desired information relating to the surface of the skin. A polarizer may be suitable for selecting the coherent electromagnetic radiation reflected from the surface of the skin and may be suitable for deselecting parts of the coherent electromagnetic radiation having interacted with skin layer such as the dermis or deeper layers.

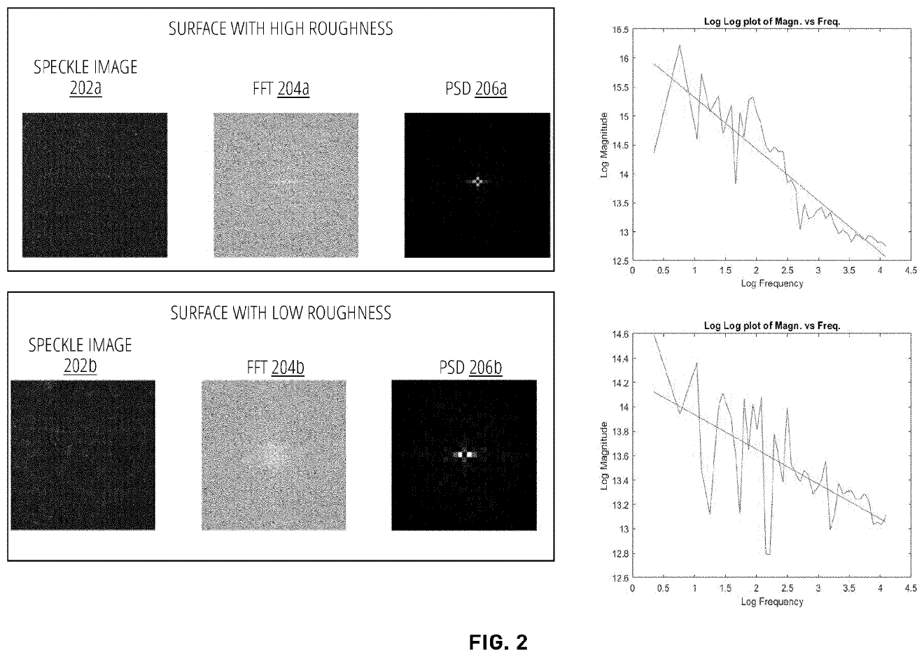

The 106 may be associated with a field of view as indicated by the two lines originating from the 106. The 106 may have a field of view between 10°×10° and 75°×75°, preferably 55°×65°. The 106 may have a resolution below 2 megapixel, preferably between 0.3 megapixel and 1.5 megapixel. Examples for the Speckle image can be found in FIG. 2.

The field of illumination may correspond at least partially to the field of view. At least a fraction of the field of view associated with the 106 may be independent of illumination with coherent electromagnetic radiation. Followingly, the Speckle image may show at least in parts the object under illumination with coherent electromagnetic radiation.

Further, the Speckle image may be provided to and/or received by a processor 108. The processor 108 may comprise one or more processors. The processor 108 may determine the surface roughness measure based on the Speckle image. The processor 108 may determine the surface roughness measure as described within the context of FIG. 2.

All system components may be part of a device, eg a device for measuring a surface roughness. In other embodiments, the system components may be separated between a plurality of devices. For example, the processor 108 may be a server, whereas the illumination source 104 and the 106 may be part of one device such as a mobile electronic device. The 106 may provide the Speckle image to the processor 108. The processor 108 may provide the surface roughness measure to a device for displaying the surface roughness measure and/or a device for processing the surface roughness measure. In an example, the device comprising the 106 and the illumination source 104 may further comprise a display for displaying the surface roughness measure and/or a surface roughness processor configured for processing the surface roughness measure further. Optionally, the device may comprise the processor 108.

FIG. 2 illustrates an example for determining a surface roughness measure. The surface roughness measure is determined based on the speckle images 202a, 202b. Examples for speckle images 202a, 202b are shown in FIG. 2. The Speckle image 204 may be cropped to a predefined size. The speckle images 202a, 202b may be transformed by means of Fourier transformation. The result of fourier transforming the speckle images 202a, 202b can be referred to as Fourier plots 204a, 204b. In particular, the Fourier plots may be obtained by means of Fast Fourier transform (FFT). The Fourier plots 204a, 204b may represent the speckle images 202a, 202b in the frequency domain. The Fourier plots 204a, 204b may represent the distribution of frequencies associated with the speckle images 202a, 202b. Followingly, the Fourier plots 204a, 204b may comprise the magnitude of frequencies associated with the speckle images 202a, 202b. The Fourier plots 204a, 204b, may be further transformed into power spectral density (PSD) plots 208. The Fourier plots 204a, 204b may be transformed into the PSD plots 206a, 206b by multiplying the magnitudes of the respective Fourier plots 204a, 204b with its conjugate. Radial averaging with respect to a predefined point such as the center point of a quadratic image may result in the double logarithmic magnitude versus frequency plots as it can be seen on the right side of FIG. 2. Radial averaging may refer to averaging values with the same distance to the predefined point. Determining the surface roughness based on PSD is advantageous since the PSD may take vertical and lateral features into account. This provides an in-depth picture of a surface roughness associated with a surface structure. Followingly, a realistic description of the surface of the object can be achieved. Further, an estimation on the distribution of surface irregularities is enabled.

The double logarithmic plotting may be used to visualize the fractal dimension. The fractal dimension may be an example for a surface roughness measure. The fractal dimension may be determined by fitting the double logarithmic magnitude versus frequency plots associated with the speckle images 202a, 202b with a linear function. In particular, the fractal dimension may be determined as the slope of the linear function fitted to the double logarithmic magnitude versus frequency plots associated with the speckle images 202a, 202b. A high surface roughness may correspond to a high fractal dimension. A low surface roughness may correspond to a low fractal dimension.

Additionally or alternatively, the surface roughness measure may comprise one or more parameters of an autocorrelation function associated with the speckle images 202a, 202b. The autocorrelation function may be obtained by inverse Fourier transform of the PDS plot 208. The autocorrelation function may be defined as follows:

$$R ( {τ}_{x} , {τ}_{y} ) = {σ}^{2} {e}^{- {( \frac{‖ {x…x}^{′} ‖}{ξ} )}^{2 α}}$$

The parameters τ, σ and ξ may be further examples for surface roughness measures. ξ may be referred to as the lateral correlation length. σ may be referred to the standard deviation of the height associated with surface features of the object. α may be referred to as roughness exponent. A high ξ may reflect a low surface roughness, a high a may reflect a high surface roughness and a high σ may reflect a high surface roughness.

Further examples for surface roughness measures may be speckle contrast, speckle modulation, speckle size or the like. These examples are readily available from the speckle images 202a, 202b.

Speckle contrast Kᵢⱼ may refer to a ratio of a standard deviation of intensity values σᵢⱼ, preferably associated with a predefined area of the speckle images 202a, 202b, to a mean of the respective intensity values I̅ᵢ̅ⱼ̅. Speckle contrast Kᵢⱼ may be defined according to the following equation:

$${K}_{ij} = \frac{{σ}_{ij}}{{\overline{I}}_{ij}}$$

Speckle modulation M may be calculated based on the following formula:

$$M = \frac{1}{N} {\sum }_{ij} \frac{{I}_{ij}^{max} - {I}_{ij}^{min}}{{I}_{ij}^{max} + {I}_{ij}^{min}} ,$$

wherein N may refer to the total number of predefined areas of the speckle images 202a, 202b, the indexes i and j may refer to pixel numbers and thus, may define the predefined area of the speckle images 202a, 202b and wherein

$${I}_{ij}^{max}$$

may refer to the maximum intensity value associated with the predefined area of the speckle images 202a, 202b and wherein

$${I}_{ij}^{min}$$

may refer to the minimum intensity value associated with the predefined area of the speckle images 202a, 202b.

The speckle size may be calculated for example by multiplying the number of pixels with the size of the pixels. In some embodiments, the speckle size may be averaged over a part of the one or more speckle images 202a, 202b and/or over the full one or more speckle images 202a, 202b.

Another embodiment for determining a surface roughness measure may comprise providing at least one of the speckle images 202a, 202b to a data-driven model such as a convolutional neural network (CNN). The data-driven model may receive at least one of the speckle images 202a, 202b at an input layer. The data-driven model may further comprise one or more hidden layers and an output layer. The speckle images 202a, 202b may be of a predefined size. The input layer may be specified according to the predefined size of the speckle images 202a, 202b. The layers of the data-driven model may be connected. Hence, the speckle images 202a, 202b may be passed through the layers. In particular, the pixel values associated with the speckle images 202a, 202b may pass through the layers of the data-driven model. While the pixel values may pass through the layers of the data-driven model, the pixel values may be allowed to interact with each other and/or may be combined, preferably non-linearly. Additionally or alternatively, the pixel values may be transformed. Preferably the pixel values may be transformed into an indication of the surface roughness measure by the data-driven model. The indication of the surface roughness measure may comprise the surface roughness measure and/or the surface roughness measure may be derivable from the indication of the surface roughness measure. Followingly, the surface roughness measure may be received from the data-driven model and/or the data-driven model may provide the surface roughness measure.

In an embodiment, the data-driven model may be configured for providing the surface roughness measure, in particular by transforming the indication of the surface roughness measure into a surface roughness measure.

Using a data-driven model may be advantageous since these models may learn correlations being non-obvious or may reflect correlations between different factors an expert would consider easily. So, the use of a data-driven model may reduce the time invest while achieving accuracies exceeding whitebox models.

In an embodiment, the data-driven model may provide the indication of the surface roughness measure and/or the indication of the surface roughness measure may be received from the data-driven model. From the indication of the surface roughness measure, the surface roughness measure may be derivable by means of a mathematical operation and/or by means of a look up table. For example, the data-driven model may be classifier classifying speckle images 202a, 202b into different groups of surface roughness measures. Hence, the output may indicate the group label. The group label may indicate the surface roughness measure. The relation between the group label and the surface roughness measure may be specified eg by the look up table. Other embodiments for establishing the relation between the surface roughness measure and the indication of the surface roughness measure may be feasible.

For the purpose described above, the data-driven model may be parametrized and/or trained according to a training data set. The training data set may comprise a plurality of speckle images 202a, 202b and corresponding surface roughness measures and/or indications of the surface roughness measure. The surface roughness measure and/or the indication of the surface roughness measure may refer to a label associated with the speckle images 202a, 202b. Parametrizing may be a prerequisite for training the data-driven model. The data-driven model may be trained based on the parametrizing of the data-driven model.

Another embodiment for determining a surface roughness measure may comprise providing the speckle images 202a, 202b to a physical model. The physical model, preferably, may reflect physical phenomena in mathematical form, e.g., including first-principles models. A physical model may comprise a set of equations that describe an interaction between the object, in particular the surface of the object and the coherent electromagnetic radiation thereby resulting in a surface roughness measure. For example, the physical model may comprise and/or combine at least one of the relations associated with the speckle contrast, the speckle modulation, the speckle size, the fractal dimension or a combination thereof. The physical model may be a white box model. The physical model may transform the speckle images 202a, 202b into a surface roughness measure. The physical model may combine the relations described above linearly, eg to introduce a weighting between the speckle contrast, the speckle modulation, the speckle size, the fractal dimension or the like. Some of the factors described above may be related closely while others may be related loosely to the surface roughness measure. Hence, weighting may reflect these relations which results in a higher accuracy. This in turn enables the reliable determination of the surface roughness because one of the factors may not be sufficient for significant results.

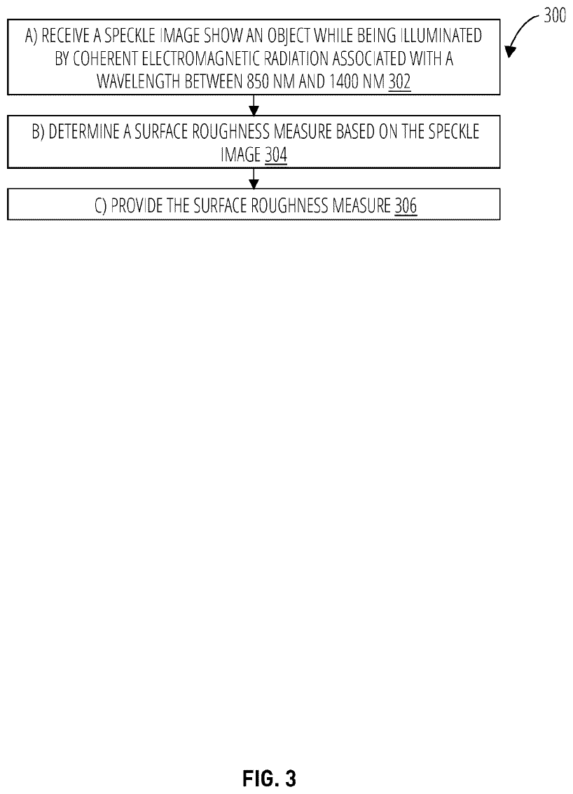

FIG. 3 illustrates an example embodiment of a method for measuring a surface roughness associated with an object 300.

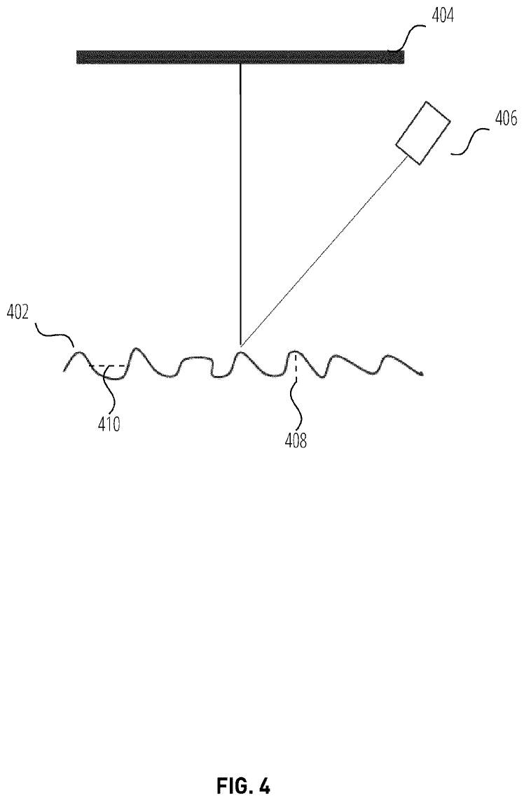

In block 302 the speckle image may be received. The speckle image may be received from and/or generated by a camera as described within the context of FIG. 1 and FIG. 4. The coherent electromagnetic radiation may be emitted from the illumination source as described in the context of FIG. 1. Further, the camera and the illumination source may be part of one device and/or system. The generation of the speckle image may be initiated by the user of the mobile electronic device. The user may desire to evaluate the surface roughness associated with the user. The user may operate an application. The application may trigger the generation of the speckle image, the receiving of the speckle image, the determining of the surface roughness measure, the providing of the surface roughness measure or a combination thereof.

Prior to determining the surface roughness measure in block 304, the speckle image may be cut to a predefined size. Thereby, background may be removed and the degree of speckles associated with the object may be increased. Speckles associated with the object, in particular the living organism such as a human may refer to speckles caused by coherent electromagnetic radiation illuminating at least a part of the object. Preferably, the predefined size may reflect the spatial extent of the object in the speckle image. For example, where the object may be associated with a circular shape of the object, the speckle image of the predefined size may be of circular shape following the circular shape of the object or an arbitrarily shaped speckle image comprising the area associated with the circular shape of the object.

In block 304 the surface roughness measure may be determined as described within the context of FIG. 2 Preferably, the surface roughness measure may be determined by a processor as described within the context of FIG. 1. The processor, the camera and the illumination source may be part of one device and/or system.

In block 306 the surface roughness measure may be provided. For example, the surface roughness measure may be provided to an application of a mobile electronic device. Further, the application may be configured for initiating the determining of the surface roughness measure and/or for initiating the generating of the speckle image. The application may display the surface roughness measure, in particular the value of the surface roughness measure for example to the human. The human may be the user and/or owner of the mobile electronic device. Hence, surface roughness measure, in particular the value of the surface roughness measure may be provided to the human. The application may further process the surface roughness measure to derive properties of the human skin.

FIG. 4 illustrates an embodiment of a surface 402 associated with an object. The surface 402 may comprise a plurality of surface features. Surface features may be lateral surface features 410 and/or vertical surface features 408. The lateral surface feature 410 may be quantified according to the dashed line indicating a length of a sink on the surface 402. The vertical surface features 408 may be quantified according to the dashed line indicating a height of an uplift on the surface 402. This surface 402 may be illuminated by coherent electromagnetic radiation emitted from the illumination source 406 as described in the context of FIG. 1 and FIG. 3. A speckle image may be generated while the surface may be illuminated by coherent electromagnetic radiation with the camera 404 as described in the context of FIG. 1 and FIG. 3.

The present disclosure has been described in conjunction with preferred embodiments and examples as well. However, other variations can be understood and effected by those persons skilled in the art and practicing the claimed invention, from the studies of the drawings, this disclosure and the claims. Notably, in particular, the any steps presented can be performed in any order, i.e. the present invention is not limited to a specific order of these steps. Moreover, it is also not required that the different steps are performed at a certain place or at one node of a distributed system, i.e. each of the steps may be performed at different nodes using different equipment/data processing.

As used herein “determining” also includes “initiating or causing to determine”, “generating” also includes “initiating and/or causing to generate” and “providing” also includes “initiating or causing to determine, generate, select, send and/or receive”. “Initiating or causing to perform an action” includes any processing signal that triggers a computing node or device to perform the respective action.

In the claims as well as in the description the word “comprising” does not exclude other elements or steps. The indefinite article “a” or “an” and the definite article “the” does not exclude a plurality. In particular, indefinite article “a” or “an” may be replaced with one or more and the definite article “the” may be replaced with the one or more. A single element or other unit may fulfill the functions of several entities or items recited in the claims. The mere fact that certain measures are recited in the mutual different dependent claims does not indicate that a combination of these measures cannot be used in an advantageous implementation.

Any disclosure and embodiments described herein relate to the methods, the systems, devices, the computer program element lined out above and vice versa. Advantageously, the benefits provided by any of the embodiments and examples equally apply to all other embodiments and examples and vice versa.

1. A method for measuring a surface roughness associated with an object, the method comprising:
a) receiving a speckle image showing an object while being illuminated by coherent electromagnetic radiation associated with a wavelength between 850 nm and 1400 nm,
b) determining a surface roughness measure based on the speckle image, and
c) providing the surface roughness measure.

2. The method of claim 1, wherein the coherent electromagnetic radiation is associated with a wavelength between 900 nm and 1000 nm and/or wherein the coherent electromagnetic radiation is associated with a wavelength between 1100 nm and 1200 nm and/or wherein the coherent electromagnetic radiation is associated with a wavelength between 1340 nm and 1440 nm.

3. The method of claim 1, wherein determining the surface roughness measure based on the speckle image refers to determining the surface roughness measure based on a speckle pattern in the speckle image.

4. The method of claim 1, wherein the speckle image is generated by a camera comprising an image sensor, a lens and a polarizer.

5. The method of claim 1, wherein the coherent electromagnetic radiation is a patterned coherent electromagnetic radiation and/or wherein the coherent electromagnetic radiation comprises one or more light beams.

6. The method of claim 1, wherein the object is a human and the surface roughness is associated with the skin of the human.

7. The method of claim 6, wherein the human has generated the speckle image and/or initiated the generation of the speckle image.

8. The method of claim 1, wherein a distance between the object and a camera used for generating the speckle image is between 10 cm and 1.5 m and/or wherein a distance between the object and an illumination source used for illuminating the object is between 10 cm and 1.5 m.

9. The method of claim 1, wherein the surface roughness measure is determined based on the speckle image by providing the speckle image to a model and receiving the surface roughness measure from the model.

10. The method of claim 1, further including reducing the speckle image to a predefined size prior to determining the surface roughness measure.

11. The method of claim 1, wherein the surface roughness measure is determined by a mobile electronic device and/or wherein the speckle image is generated with a camera of a mobile electronic device.

12. The method of claim 1, wherein the speckle image is associated with a resolution of less than 5 megapixel.

13. A device or system for measuring a surface roughness, the system or device comprising:
(a) an illumination source configured for illuminating an object with coherent electromagnetic radiation associated with a wavelength between 850 nm and 1400 nm,
(b) a camera configured for generating a speckle image showing an object under illumination with the coherent electromagnetic radiation associated with a wavelength between 850 nm and 1400 nm, and
(c) a processor configured for receiving the speckle image from the camera and determining a surface roughness measure based on the speckle image and providing the surface roughness measure.

14. A method of using Use of a surface roughness measure as obtained by the method according to claim 1, the method comprising using the surface roughness measure for evaluating the surface roughness of a human.

15. A non-transitory computer-readable storage medium, the computer-readable storage medium including comprising instructions, wherein when the computer-readable storage medium is executed by a computer, the instructions cause the computer to:
a) receive a speckle image showing an object under illumination with coherent electromagnetic radiation associated with a wavelength between 850 nm and 1400 nm,
b) determine a surface roughness measure based on the speckle image, and
c) provide the surface roughness measure.

16. A method of using a surface roughness measure as obtained by the device or system according to claim 13, the method comprising using the surface roughness measure for evaluating the surface roughness of a human.
